(19) United States
(12) Patent Application Publication
(10) Pub. No.: US 2018/0214227 A1
(43) Pub. Date: Aug. 2, 2018

(54) SUCTION MOUNTED MICROSURGICAL INSTRUMENT

(71) Applicant: SANDEEP SOOD, TROY, MI (US)

(72) Inventor: SANDEEP SOOD, TROY, MI (US)

(21) Appl. No.: 15/420,103

(22) Filed: Jan. 31, 2017

(51) Int. Cl. A61B 34/00; A61B 17/295; A61B 17/3201; A61M 1/00

(52) U.S. Cl. CPC A61B 34/72; A61B 17/295; A61B 2017/00345; A61M 1/0086; A61B 17/3201

(57) ABSTRACT

A microsurgical instrument that can slide over a surgical suction instrument and comprises a grasper or a micro-scissor for cutting at the distal end, a lever at the proximal end to actuate the jaws of the grasper or the blades of the micro scissor. The disclosed microsurgical instrument may be locked to the surgical suction instrument or may be rotated on the long axis on the surgical suction instrument. The disclosed instrument can be placed by sliding over the surgical suction instrument without the need for disconnecting the surgical suction instrument from the suction hose.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of brain surgery and more specifically to microsurgical instruments that can be mounted over a surgical suction instrument. The invention is designed to improve micro-neurosurgery of the brain.

Surgery of the brain is done with a microscope that provides visualization and magnification. The microscope is generally fixed in one position and to move to the area of interest the surgeon physically moves the microscope head holding the handles provided for this purpose. Once the surgeon is satisfied with the position of the microscope, he generally performs the surgery looking through the microscope at the tissue using one instrument in one hand and the other instrument in the other hand. A surgical suction instrument (referred to subsequently as surgical suction), is generally a device made of a metal that has a proximal end and a distal end. At the proximal end the surgical suction is connected with a hose to a low-pressure vacuum suction system. The surgeon generally holds the surgical suction in the left hand and places the distal end of the surgical suction in the surgical field so that the suction at the distal end removes blood and debris from the surgical field to keep the surgical field clear. A second instrument is used in the right hand. This may be a dissecting instrument, grasping instrument, cutting instrument or any of the myriad of instruments that are used to manipulate tissue as deemed necessary by the surgeon. The two instruments, surgical suction in the left hand and the instrument in the right hand are used in conjunction with each other. The distal end of the surgical suction in-addition to keeping the field clear of blood is also used to provide counter pressure on the tissue to aid dissection with the second instrument in the right hand. This type of two handed or bimanual ability to manipulate the tissue with two instruments is critical to the objective of surgery which is to dissect and separate abnormal tissue from normal tissue. Bleeding during surgery is controlled with an instrument called bipolar cautery forceps, which is used when needed by replacing the instrument in right hand. During the course of surgery, the surgeon may sometimes need to use a different instrument in the left hand, such as a grasper or a micro-scissor. He may accomplish this by removing the surgical suction from the field of surgery and handing it to an assistant, and then take the desired instrument in the left hand and continue with the surgery. During this time the field of surgery may become obscured by blood requiring him to hand back the instrument, replace it with surgical suction, clear the field and then take the instrument back. This is not only frustrating but also increases the duration of surgery. Further working in a surgical field partially obscured by blood may be dangerous and risk injuring sensitive structures.

Rafferty et al U.S. Pat. No. 387,046 described a medical instrument attached to a suction. However, each time a different medical instrument is required, the suction hose from the medical instrument has to be disconnected and connected to the different medical instrument. This is tedious and cumbersome for the surgeon.

Whipple U.S. Pat. No. 4,522,206 describe a cutting suctioning instrument for use in knee surgery which has the same limitation.

Teichtmann U.S. Pat No. 8,603,133 B2 relates to a medical instrument which has a distal jaw member that can rotate with respect to a rigid jaw and has a handle positioned on the proximal end. Again, this device does not provide the ability to have a suction.

Tsuchiya et al U.S. Pat. No. 8,652,132 B2 describe a grasping device that includes jaws attached to a distal portion of a sheath rotatable around a rotation axis. Again, this device does not have ability to provide suction through it.

Kalmann et al U.S. Pat. No. 9,066,744 B2 describes a surgical instrument for grasping and culling tissue. The device does not have ability to provide suction to keep the field clear.

Stoddard et al U.S. Pat. No. 9,161,769 B2 is an endoscopic instrument with jaw members at end of inner and out shaft members with a handle at the other end. It can grasp tissue for endoscopic surgery but does not have ability to provide suction.

In short, in prior art, though grasping microsurgical instruments, and surgical suction instrument devices exist separately and even together, there is no microsurgical instrument that can be placed by sliding the microsurgical instrument on a surgical suction instrument without disconnecting the surgical suction instrument from the suction hose, and provide dual function of suction and grasping or a microsurgical instrument that can be placed by sliding the microsurgical instrument on a surgical suction instrument without disconnecting the surgical suction instrument from the suction hose, and provide dual function of suction and cutting with a micro-scissor in the manner of the present invention.

### BRIEF SUMMARY OF THE INVENTION

The primary object of the invention is to be able to do two handed dissection of tissues during brain surgery without the need to forgo surgical suction in the left hand.

Another object of the invention is to be able to use a grasper or a micro-scissor for cutting in the left hand and be able to keep the field of surgery clear with surgical suction and still be able to use another instrument in the right hand for bimanual surgery.

Another object of the invention is to be able to place the grasper microsurgical instrument or the micro scissor microsurgical instrument on the surgical suction by sliding the microsurgical instrument over the surgical suction without disconnecting the surgical suction from the suction hose.

Another object of the invention is to be able to activate the grasper microsurgical instrument to grasp tissue or to activate the micro-scissor instrument to cut using a lever conveniently located on the assembly.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a microsurgical instrument for two handed surgical dissections during brain surgery comprising: A microsurgical instrument that can slide over a surgical suction and perform the task of grasping tissue or cutting tissue with a micro scissor and be activated by a conveniently located lever on the assembly and at the same time keep the field clear through the action of the surgical suction.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1A shows the perspective view of three of the five components of the invention.

FIG. 1B shows another perspective view of the components shown in FIG. 1A.

FIG. 1C shows the sectional view along the long axis of components shown in FIG. 1A.

FIG. 2A shows the perspective view of the other two components of the invention.

FIG. 2B shows the sectional view along the long axis of the components shown in FIG. 2A.

FIG. 3A shows the perspective view of the assembled invention.

FIG. 3B shows the sectional view along the long axis of the assembled invention shown in FIG. 3A.

FIG. 4A is the sectional view along the long axis of the present invention in use as it slides over a surgical suction, showing one of the modalities of use with the jaw closed. A′-A and B′-B are cross sectional views.

FIG. 4B is the sectional view along the long axis of the present invention in use as it slides over a surgical suction, showing another of the modalities of use with the jaw open.

FIG. 5 shows the perspective view of one alternative modality of the components of the invention with sharp blades between the jaws to function as a micro-scissor.

FIG. 6A shows the sectional view along the long axis of the alternative modality of the component of the invention shown in FIG. 5 functioning as a micro-scissor with jaw open.

FIG. 6B shows the sectional view along the long axis of the alternative modality of the component of the invention shown in FIG. 5 functioning as a micro-scissor with jaw closed.

FIG. 7A shows the perspective view of another alternative modality of the components of the invention with one side of the corresponding jaws sharpened to function as a micro-scissor.

FIG. 7B shows the sectional view along the long axis of the alternative modality of the components of the invention shown in FIG. 7A as a micro-scissor with the jaw closed.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

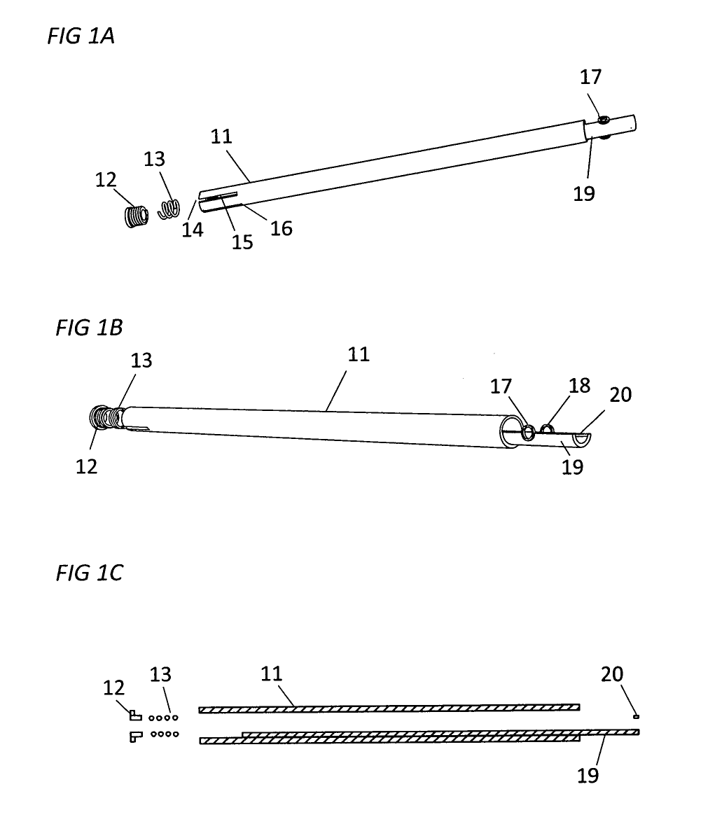

FIG. 1A shows the perspective view of three of the five components of the invention comprising a tube 11, a nut 12 and spring 13. The tube 11 has a proximal end and a distal end on the long axis. At the proximal end 14 of tube 11 there are two slots 15 and 16 and on the inside of the tube at this end are threads to receive the hollow nut 12 with a central opening, and, spring 13. The nut 12 has threads on the outside complimentary to the threads on the inside of the tube 11 at the proximal end 14. On the distal end of the long axis of the tube 11, there are two rings 17 and 18 on the half cylinder 19 which is placed inside the tube 11 and attached to it by means such a soldering or welding or be made as a single mold such that a part of the half cylinder 19 extends beyond the distal end of tube 11 as shown in FIG. 1B on the right side. On distal end, the half cylinder 19 has a flat bar 20. FIG. 1C shows the sectional view along the long axis of tube 11 of the components shown in FIG. 1A.

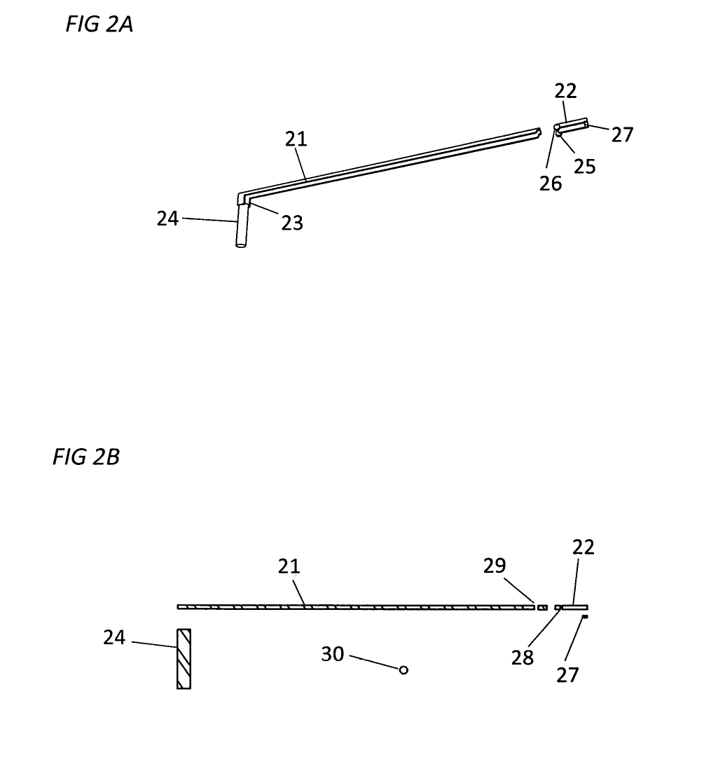

FIG. 2A shows the perspective view of the other components of the invention comprising half cylinder 21 complimentary to the half cylinder 19 shown in FIG. 1A, and the jaw 22. The half cylinder 21 has two wings 23 at proximal end that can slide in the slots 15 and 16 of tube 11. The wings 23 are attached to a lever 24. The distal end of the half cylinder 21 is contoured so that the component 22 aligns with cylindrical projections 25 and 26 onto this end of component 21. The cylindrical projections are such that they fit into the rings 17 and 18 of component 19, and can rotate along the common central axis of the rings 17 and 18. Component 22 has a bar 27 at the end opposite to the cylindrical projections 25 and 26 to align with the bar 20 on component 19. There is a hole 28 on the component 22 on the same end as the cylindrical projections 25 and 26. There is also a hole 29 on component 21 on the side it is contoured to align with the cylindrical projections 25 and 26. The ring 30 goes through the holes 28 and 29 so that it keeps component 22 attached loosely to component 21. FIG. 2B is the section view along the long axis of half cylinder 21 of the components of the invention shown in FIG. 2A

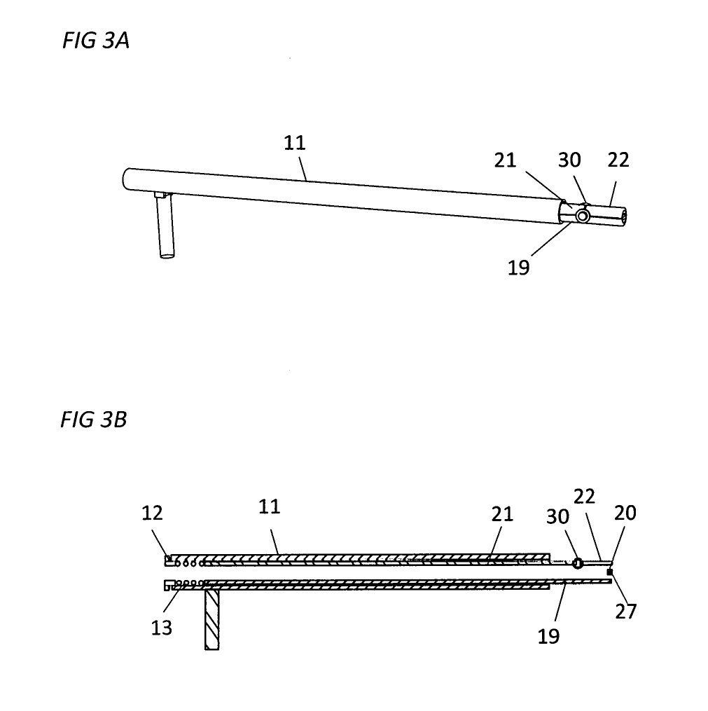

FIG. 3A shows the perspective view of the assembled invention. FIG. 3B is the sectional view along the long axis of the assembled invention shown in FIG. 3A.

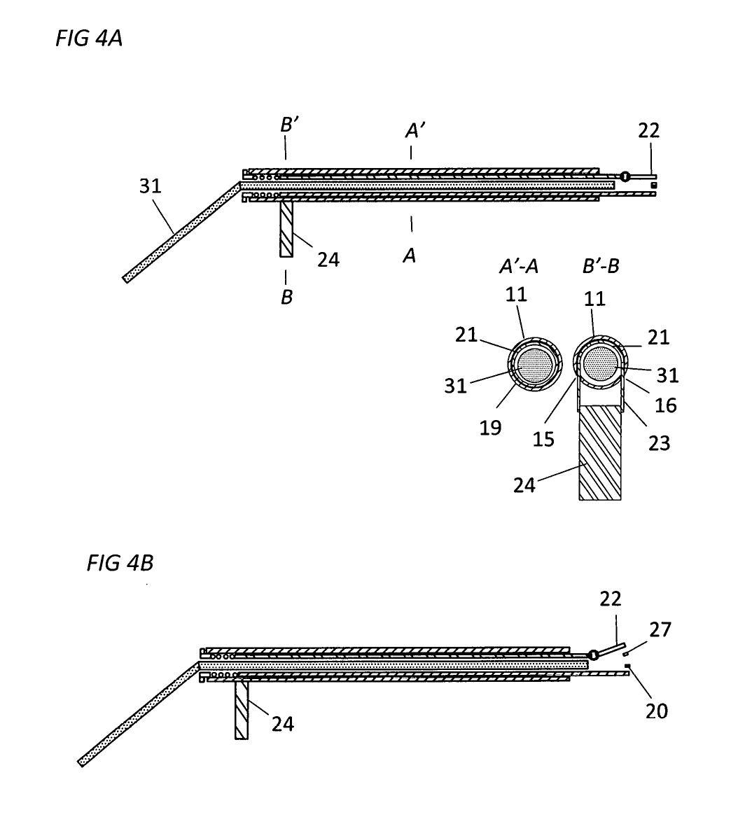

FIG. 4A shows the sectional view along the long axis of the assembled invention in one modality of use of the invention. A′-A and B′-B represent the cross-sectional views of the assembled invention across the corresponding markings on sectional view of the assembled invention shown in FIG. 4A. The assembled invention slides on the long axis on the surgical suction 31 through the central opening in the nut 12 seen in FIG. 3B. The end of the surgical suction 31 that is not inside the assembled invention is connected through a hose, not shown in the figure, to low pressure vacuum suction. There may be a locking mechanism as a screw or a slot to secure the assembled invention to the surgical suction. Alternatively, the assembled invention can be rotated, on the long axis, on the surgical suction to the desired position for use of the grasper. The surgical suction that is connected with a hose to low pressure vacuum suction and with the assembled invention as described above, is held in the surgeon's left hand for use during surgery. During surgery, if a need arises to grasp tissue, the lever 24 is pulled with a finger such that component 21 slides in tube 11 on component 19 and by its attachment to component 22 through ring 30 lifts the component 22 rotating it at the axis of the projections 25 and 26 that are in the rings 17 and 18. As shown in the sectional view along the long axis of the assembled invention in FIG. 4B, this causes the component 22 to rotate as a result of which the bar 27 is moved away from bar 20. At this time the surgeon moves the surgical suction, with the invention on it, forwards so that the tissue to be grasped touches bar 20. On releasing the lever 24, the spring 13 pushes the component 21 back into its original position, forcing the component 22 back to the original position, causing the bar 27 of component 22 come into apposition with bar 20 of component 19 hence grasping the tissue.

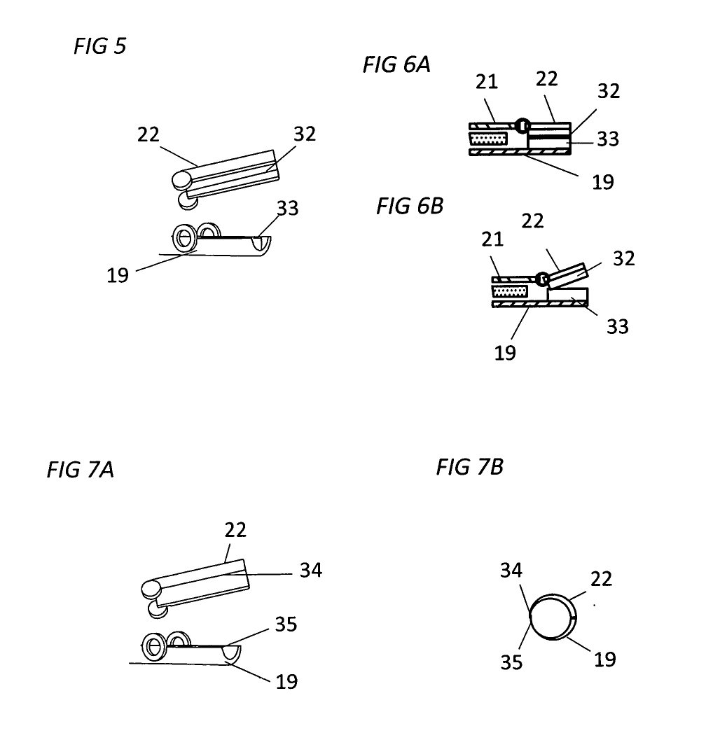

In another embodiment of the invention shown in FIG. 5, instead of the bar 27 on the component 22, there is a central sharp blade 32 on component 22 and sharp blade 33 on component 19, such that when the lever 24 is pulled, blades move apart as shown in sectional view FIG. 6A and on releasing the lever 24 they come together, as shown in sectional FIG. 6B, resulting in scissor action and hence cutting the tissue between the blades.

In yet another embodiment of the invention shown in FIG. 7A, one of the sides of component 22 such as 34 has a sharp edge. The complimentary edge 35 of the component 19 also has a sharp edge so that any tissue between them during use would be cut as the component 22 apposes to component 19, as shown in sectional view FIG. 7B, at the time lever 24 is released. This would function as a micro-scissor for cutting.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1. A microsurgical instrument for surgery that comprises:
A proximal end and a distal end on the long axis;
A central opening at the proximal end through which the microsurgical instrument can slide on the long axis over a surgical suction instrument;
A grasper or a micro-scissor at the distal end;
A lever at the proximal end that can actuate the gasper or micro-scissor at the distal end;

2. A microsurgical instrument for surgery in claim 1, which may be secured to the surgical suction instrument by a locking mechanism.

3. A microsurgical instrument for surgery in claim 1, when in use with the surgical suction instrument provides dual function of suction and grasping or of suction and cutting with micro-scissor.

4. (canceled)

5. A microsurgical instrument for surgery in claim 1, which may be rotated along the long axis on the surgical suction instrument.
